(19) United States
(12) Patent Application Publication
(10) Pub. No.: US 2015/0241319 A1
(43) Pub. Date: Aug. 27, 2015

(54) SWAB INTERFACE FOR A MICROFLUIDIC DEVICE

(71) Applicants: Thomas N. CHIESL, (US); Steven G. HAUPT, San Diego, CA (US); Ronald K. BERGOLD, Mission Viejo, CA (US); Bradley J. SARGENT, Mission Viejo, CA (US); Steven A. HOFSTADLER, Vista, CA (US); IBIS BIOSCIENCES INC., Carlsbad, CA (US)

(72) Inventors: Thomas N. Chiesl, Hurcules, CA (US); Steven G. Haupt, San Diego, CA (US); Ronald K. Bergold, Mission Viejo, CA (US); Bradley J. Sargent, Mission Viejo, CA (US); Steven A. Hofstadler, Vista, CA (US)

(21) Appl. No.: 14/431,628

(22) PCT Filed: Sep. 26, 2013
(86) PCT No.: PCT/US13/61921

§ 371 Date: Mar. 26, 2015

Related U.S. Application Data
(60) Provisional application No. 61/705,967, filed on Sep. 26, 2012.

(51) Int. Cl. G01N 1/10; B01L 3/00; G01N 1/38; A61F 13/38

(52) U.S. Cl. CPC G01N 1/10; A61F 13/38; B01L 3/508; B01L 3/502; B01L 3/502761; G01N 1/38; B01L 2200/06; B01L 2300/04; B01L 2300/0636; B01L 2300/087; G01N 2001/1056

(57) ABSTRACT

The present disclosure relates to a swab port device. In particular, the present disclosure relates to swab port device that interfaces a swab to a microfluidic device, methods of bonding the swab port to the device, methods of recirculating liquid over the swab, and an enclosure system to seal the device.

The present application claims priority to U.S. Provisional Patent Application Ser. No. 61/705,967, filed Sep. 26, 2012, the disclosure of which is herein incorporated by reference in its entirety.

### FIELD OF THE INVENTION

The present disclosure relates to a swab port device. In particular, the present disclosure relates to swab port device that interfaces a swab to a microfluidic device, methods of bonding the swab port to the device, methods of recirculating liquid over the swab, and an enclosure system to seal the device.

### BACKGROUND OF THE INVENTION

Many research and clinical assays utilize sample collection via swabs or other disposable sample collection devices. For example, genetic testing, infectious disease testing (e.g., swabs from body orifices), etc. all utilize sample collection devices. The samples on the swabs are generally transferred to an analysis device or component for further testing.

Currently, many steps and accessories are required to process swabs including scissors, tubes, vortexers, and centrifuges. Additional devices and methods for streamlining removal of samples from swabs are needed.

### SUMMARY OF THE INVENTION

The present disclosure relates to a swab port device. In particular, the present disclosure relates to swab port device that interfaces a swab to a microfluidic device, methods of bonding the swab port to the device, methods of recirculating liquid over the swab, and an enclosure system to seal the device.

Embodiments of the present invention provide a swab port device, comprising: at least one body structure comprising one or more surfaces that define a first cavity having upper and lower portions and a second cavity having upper and lower portion; and a lid configured to seal the first and second cavities. In some embodiments, the first cavity is sized to accept a sample collection swab. In some embodiments, the first and second cavities are in fluid communication. In some embodiments, the first cavity comprises a volume capacity of about 300 μL (e.g., 0.50 to 5000 μL, 50 to 1000 μL, 50 to 500 μL, etc.). In some embodiments, the body structure further comprises a plurality of protrusions (e.g., protrusions or feet of different sizes or shapes), e.g., configured to align the device to holes in an analysis device. In some embodiments, the lid comprises a lid sealing component and a gasket component. In some embodiments, the first cavity comprises a neck. In some embodiments, the lid is integrated into the swab port device.

In some embodiments, the first cavity comprises one or more interior protrusions (e.g., teeth) to facilitate removal of material from the swab when the swab comes in contact with the protrusions, for example, by sheering, squeegee, dislodgement, or any other force.

Further embodiments provide a system, comprising: a swab port device as describe herein; and an assay component (e.g., microfluidic device) in communication with the device. In some embodiments, protrusions of the device are inserted in holes in the assay component and optionally the protrusions are heat sealed or otherwise attached to the assay component. In some embodiments, the system further comprises a sample analysis component operably linked to the assay component.

In yet other embodiments, the present invention provides a method, comprising: a) contacting a swab port device comprising i) at least one body structure comprising one or more surfaces that define a first cavity having upper and lower portions and a second cavity having upper and lower portion; and ii) a plurality of protrusions protruding from the bottom of the body structure with an assay component comprising holes sized to receive the protrusions; and b) sealing the device to the assay component by applying heat that melts the protrusions to the assay component.

In still further embodiments, the present invention provides a method, comprising: contacting a system as described herein with a swab comprising a sample; optionally breaking off the end of the swab such that the portion of the swab containing the sample remains in the first cavity of the device; and circulating liquid contained in the second cavity through the first cavity (e.g., such that the sample is transferred to the liquid). In some embodiments, the method further comprises the step of contacting the liquid with said the component (e.g., microfluidic device). In some embodiments, the method further comprises the step of identifying an analyte in the sample (e.g., including but not limited to, a nucleic acid, an amino acid, a lipid, a metabolite, or a chemical analyte).

In some embodiments, provided herein is the use of a device or system as described above. In some embodiments, provided herein is the use of a device or system as described above, for the collection of chemical, biological, or environmental materials from a swab, for example, for diagnostic, screening, therapeutic, or research purposes (e.g., diagnosis of a medical condition or infection of a subject).

Additional embodiments are described herein.

### BRIEF DESCRIPTION OF THE DRAWINGS

The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 shows an exemplary swab port and lid of embodiments of the present disclosure.

FIG. 2 shows exemplary components for sealing a swab port to an analysis component.

FIG. 3 shows exemplary components for interfacing and sealing a swab port to an analysis component.

FIG. 4 shows an exemplary lid gasket combination of embodiments of the present disclosure.

FIG. 5 shown a lid/gasket interfaced with a swab port.

FIG. 6 shows a swab inserted into a swab port of embodiments of the present disclosure.

FIG. 7 show an exemplary reflux port of embodiments of the present disclosure.

FIG. 8 shows recirculation of solutions through a reflux port and recovery of nucleic acids from a swab.

FIG. 9 shows exemplary devices of embodiments of the present invention.

FIG. 10 shows an exemplary device comprising interior teeth-like protrusions to assist in the removal of materials from an inserted swab.

### DEFINITIONS

Before describing the invention in detail, it is to be understood that this invention is not limited to particular devices, systems, kits, or methods, which can vary. As used in this specification and the appended claims, the singular forms “a,” “an,” and “the” also include plural referents unless the context clearly provides otherwise. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the invention, the following terminology, and grammatical variants thereof, will be used in accordance with the definitions set forth below.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

The term “base composition” refers to the number of each residue comprised in an amplicon or other nucleic acid, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill F et al. (1998) “Polymerase recognition of synthetic oligodeoxyribonucleotides incorporating degenerate pyrimidine and purine bases” Proc Natl Acad Sci U.S.A. 95(8):4258-63), an acyclic nucleoside analog containing 5-nitroindazole (Van Aerschot et al., Nucleosides and Nucleotides, 1995, 14, 1053-1056), the purine analog 1-(2-deoxy-beta-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, 0⁶-methyl-2′-deoxyguanosine-5′-triphosphate, N²-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C. In some embodiments, the non-natural nucleosides used herein include 5-propynyluracil, 5-propynylcytosine and inosine. Herein the base composition for an unmodified DNA amplicon is notated as A_{w}GₓC_{y}T_{z}, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. A match between the calculated base composition and a single database entry reveals the identity of the bioagent.

The term “communicate” refers to the direct or indirect transfer or transmission, and/or capability of directly or indirectly transferring or transmitting, something at least from one thing to another thing. Objects “fluidly communicate” with one another when fluidic material is, or is capable of being, transferred from one object to another. For example, in some embodiments of the present invention, a swab port is in fluid communication with a reflux port.

The term “kit” is used in reference to a combination of articles that facilitate a process, method, assay, analysis or manipulation of a sample. Kits can contain instructions describing how to use the kit (e.g., instructions describing the methods of the invention), swab ports, microfluidic devices, lids, components for heat sealing, assay reagents, as well as other components. Kit components may be packaged together in one container (e.g., box, wrapping, and the like) for shipment, storage, or use, or may be packaged in two or more containers.

The term “material” refers to something comprising or consisting of matter. The term “fluidic material” refers to material (such as, a liquid or a gas) that tends to flow or conform to the outline of its container.

The term “microplate” refers to a plate or other support structure that includes multiple cavities or wells that are structured to contain materials, such as fluidic materials. The wells typically have volume capacities of less than about 1.5 mL (e.g., about 1000 μL, about 800 μL, about 600 μL, about 400 μL, or less), although certain microplates (e.g., deep-well plates, etc.) have larger volume capacities, such as about 4 mL per well. Microplates can include various numbers of wells, for example, 6, 12, 24, 48, 96, 384, 1536, 3456, 9600, or more wells. In addition, the wells of a microplate are typically arrayed in a rectangular matrix. Microplates generally conform to the standards published by the American National Standards Institute (ANSI) on behalf of the Society for Biomolecular Screening (SBS), namely, ANSI/SBS 1-2004: Microplates—Footprint Dimensions, ANSI/SBS 2-2004: Microplates—Height Dimensions, ANSI/SBS 3-2004: Microplates—Bottom Outside Flange Dimensions, and ANSI/SBS 4-2004: Microplates—Well Positions, which are each incorporated by reference. Microplates are available from a various manufacturers including, e.g., Greiner America Corp. (Lake Mary, Fla., U.S.A.) and Nalge Nunc International (Rochester, N.Y., U.S.A.), among many others. Microplates are also commonly referred to by various synonyms, such as “microtiter plates,” “micro-well plates,” “multi-well containers,” and the like

The term “molecular mass” refers to the mass of a compound as determined using mass spectrometry, for example, ESI-MS. Herein, the compound is preferably a nucleic acid. In some embodiments, the nucleic acid is a double stranded nucleic acid (e.g., a double stranded DNA nucleic acid). In some embodiments, the nucleic acid is an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

The term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N⁶-methyladenosine, aziridinylcytosine, pseudoisocytosine, 5-(carboxyhydroxyl-methyl)-uracil, 5-fluorouracil, 5-bromouracil, 5-carboxymethylaminomethyl-2-thiouracil, 5-carboxymethyl-aminomethyluracil, dihydrouracil, inosine, N⁶-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N⁶-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “system” refers a group of objects and/or devices that form a network for performing a desired objective.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present invention.

### DETAILED DESCRIPTION

The present disclosure relates to a swab port device and methods and system employing such. In particular, the present disclosure relates to swab port device that interfaces a swab to a microfluidic device, methods of bonding the swab port to the device, methods of recirculating liquid over the swab, and an enclosure system to seal the device.

Embodiments of the present invention provide a swab port on a microfluidic device such that a user can take a swab with a sample (e.g. forensics, clinical, bio warfare agent detection, environmental samples, etc.) and then break it off or otherwise separate it inside the device and close the lid to contain the swab and liquid processes encountered downstream. As a part of experiments conducted during the development of embodiments described herein, methods were developed to bond the swab port to the device (e.g., via heat staking) Thus, embodiments of the disclosure further provide methods for bonding any modular component to a microfluidic device or other system components by heat staking methods. Additional experiments developed a lid mechanism that acts as a method of sealing the swab port, while another portion of the same piece serves as a gasket to form a seal between the microfluidic card and the swab port. This has the added benefit of both providing a lid for keeping the swab and any liquid encountered downstream stay inside the swab port and that this enclosure is always attached to the card (e.g., preventing accidental loss).

The exemplary embodiments solve the problem of how to interface swabs that are traditionally used in forensics, clinical applications, and explosives to a microfluidic device by removing manual labor intensive steps from the benchtop. Embodiments further provide methods for bonding modular components to an analysis device (e.g., microfluidic device), a method for breaking the swab off inside the device, an enclosure system that functions as a gasket and a lid, and a method to reflux liquid material over the swab during microfluidic operation.

I. Devices Each aspect of the disclosure is described in further detail in the sections below. In brief these are A) Swab port; B) Heat staking modular components (e.g., swab port) to a microfluidic device; C) Enclosure system that starts as a separate component but then becomes integrated with the port as both a gasket and a lid; and D) Reflux port for out-of-plane mixing/re-circulation.

### A) Swab Port

FIG. 1 shows a schematic of a swab port 1 with the lid open, closed, a cutaway view of the piece with closed lid and a photograph of the swab port on a microfluidic card 5. The swab port 1 comprises a body 2, a lid 3, a swab insertion component 4, and a recirculation port 6 (described in more detail below).

In order to use the device a user inserts a sample collection device (e.g., swab) containing a sample into swab insertion component 4. The user then breaks off, cuts off, or otherwise separates the portion of the swab that is not enclosed by the swab insertion component 4 and closes the lid 3, as shown in the second panel of FIG. 1. The swab port 1 can then be integrated into, for example a microfluidic plate 5 for analysis.

The swab port and microfluidic device may be constructed from any suitable material. In some embodiments, pieces are made via cavity injection mold from arylic or polystyrene, although other fabrication methods and materials are specifically contemplated.

For example, in some embodiments, machining, embossing, extrusion, stamping, engraving, injection molding, cast molding, etching (e.g., electrochemical etching, etc.), or other techniques are utilized to fabricate devices. These and other suitable fabrication techniques are described in, e.g., Molinari et al. (Eds.), Metal Cutting and High Speed Machining, Kluwer Academic Publishers (2002), Altintas, Manufacturing Automation: Metal Cutting Mechanics, Machine Tool Vibrations, and CNC Design, Cambridge University Press (2000), Stephenson et al., Metal Cutting Theory and Practice, Marcel Dekker (1997), Fundamentals of Injection Molding, W. J. T. Associates (2000), Whelan, Injection Molding of Thermoplastics Materials, Vol. 2, Chapman & Hall (1991), Rosato, Injection Molding Handbook, 3^{rd} Ed., Kluwer Academic Publishers (2000), Fisher, Extrusion of Plastics, Halsted Press (1976), and Chung, Extrusion of Polymers: Theory and Practice, Hanser-Gardner Publications (2000), which are each incorporated by reference. Exemplary materials include, but are not limited to, ABS, Santoprene, HDPE, PEEK, TPE, LCP, PETG, TPV, Ultem, Nylon, Udel, PBT, PVC, Polycarbonate, Radel, polymethylmethacrylate, polyethylene, polydimethylsiloxane, polyetheretherketone, polytetrafluoroethylene, polystyrene, polyvinylchloride, polypropylene, polysulfone, polymethylpentene, and polycarbonate, among many others. In some embodiments, devices are fabricated as disposable or consumable components of mixing stations or related systems. In certain embodiments, following fabrication, system components are optionally further processed, e.g., by coating surfaces with a hydrophilic coating, a hydrophobic coating (e.g., a Xylan 1010DF/870 Black coating available from Whitford Corporation (West Chester, Pa.), etc.), or the like, e.g., to prevent interactions between component surfaces and reagents, samples, or the like.

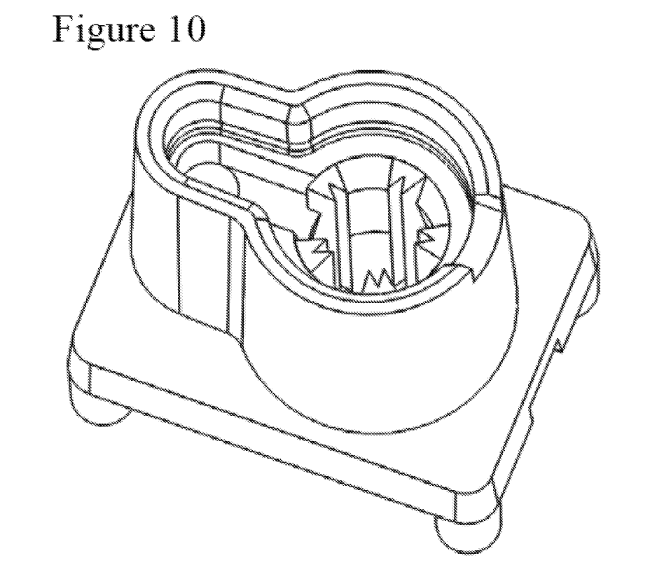

FIG. 9 shows devices of embodiments of the present invention. Panel 1) Photograph of injection molded swab port body and casted silicone enclosure part next to a penny for scale. The clear piece is made from acrylic and the opaque piece is polystyrene. 2) Top view of a polystyrene swab port body showing the main swab port hole (big hole on left) and the reflux column (right). Also visible is the channel connecting the swab port to the reflux port on the top surface to facilitate the reflux process. 3) Same injection molded design but made from acrylic material instead of styrene. 4) Bottom view of molded parts showing the small recess for the enclosure (left) and the enclosure placed inside the recess on the bottom face.

FIG. 10 shows a configuration of some embodiments of the devices where the interior surface of the cavity that receives the swab has protrusions for assisting in the removal of materials from inserted swabs. Five pairs of teeth-like protrusions are shown in FIG. 10. However, the protrusions may be of any desired shape or form to achieve the desired result (rounded, rectangular, etc.). The protrusions are preferably configured such that if a user places a swab into the port, the user can then twist the swab to help remove cellular material from the swab. Removal may be accomplished, for example, by a grinding or squeegee action.

### B) Method of Heat Staking Modular Components to Microfluidic Devices

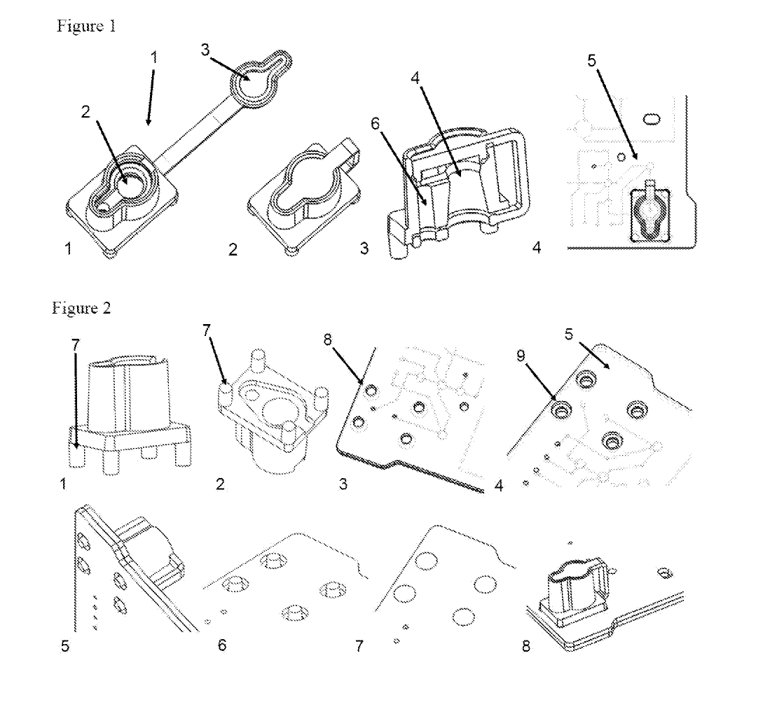

In some embodiments, the present invention provides methods for heat staking components (e.g., swab ports) to analysis components (e.g., microfluidic devices). This is illustrated, for example, in FIG. 2.

FIG. 2 shows 1) Feet features 7; 2) Bottom side view showing cavity for an optional gasket; 3) Example microfluidic device 5 topside hole 8 features correspond in position and shape to the feet on the modular component 4) Bottom side view of microfluidic device 5 showing the cavity features 9; 5) ¾ view of the backside of microfluidic device 5 with feet 7 from the swab port 1 passing through the alignment holes and cavity features; 6) lower profile view showing that the feet 7 stick beyond the bottom surface of the microfluidic device 5; 7) The feet 7 are melted into the cavity 9; 8) ¾ view of the top surface of final heat staked par (parts are shown as transparent).

In some embodiments, the modular part (e.g. swab port) that is heat staked includes features that are long enough to extend through the microfluidic part. These are referred as the feet 7 of the part and can be any shape/size (e.g., as cylinders, square pegs, triangular pegs etc.). In some embodiments, a modular part contains at least 1 foot 7 (e.g., 1, 2, 3, 4 or more feet) for heat staking. In some embodiments, components comprise 3 or 4 feet 7 for equal distribution of bonding force afterwards. The feet can be made out of any suitable material (e.g., a low temperature melting plastic such as polyacrylic or polystyrene). Feet may also include barb like structures and other features to help snap the part in place.

In some embodiments, analysis component (e.g., microfluidic device 5) card has corresponding holes 8 that match the shape of the feet on the top surface of the card and include a slightly larger cavity feature 9 on the bottom side of the microfluidic device 5. The holes and the feet can include alignment features for positioning of feet/holes and can also include the use of keyed features such that the part can only be attached one way through the position of the feet or by spatially different feet shapes (e.g. a three footed part can have a square peg in the bottom left a round peg in the bottom right and a triangle peg in the remaining position). After the feet 7 and microfluidic device 5 have been placed in contact (with the feet extending through the holes) the pieces can be optionally clamped together. The feet are then melted into the backside cavities and allowed to cool in place. The resulting device is shown in panel 8 of FIG. 2.

Optionally, a gasket can be placed in between the modular component and the microfluidic card to enhance the fluidic seal. In some embodiments, the backside cavities 9 on the microfluidic device 5 have volumes large enough to accommodate the melted feet volume. After the feet have cooled, the clamping or compression force, if used, can be removed and the modular component is bonded to the microfluidic device 5 with or with the gasket in place.

Heat staking is accomplished using any suitable method, including but not limited to, a soldering iron, a hot plate, or other device suitable for melting the feet of the swab port.

Once the clamping pressure has been removed the heat staked part is permanently bonded to the microfluidic card. The part can be removed, if desired, by re-melting the feet and pulling the swab port out. Alternatively, any desired fastening mechanism/component may be employed.

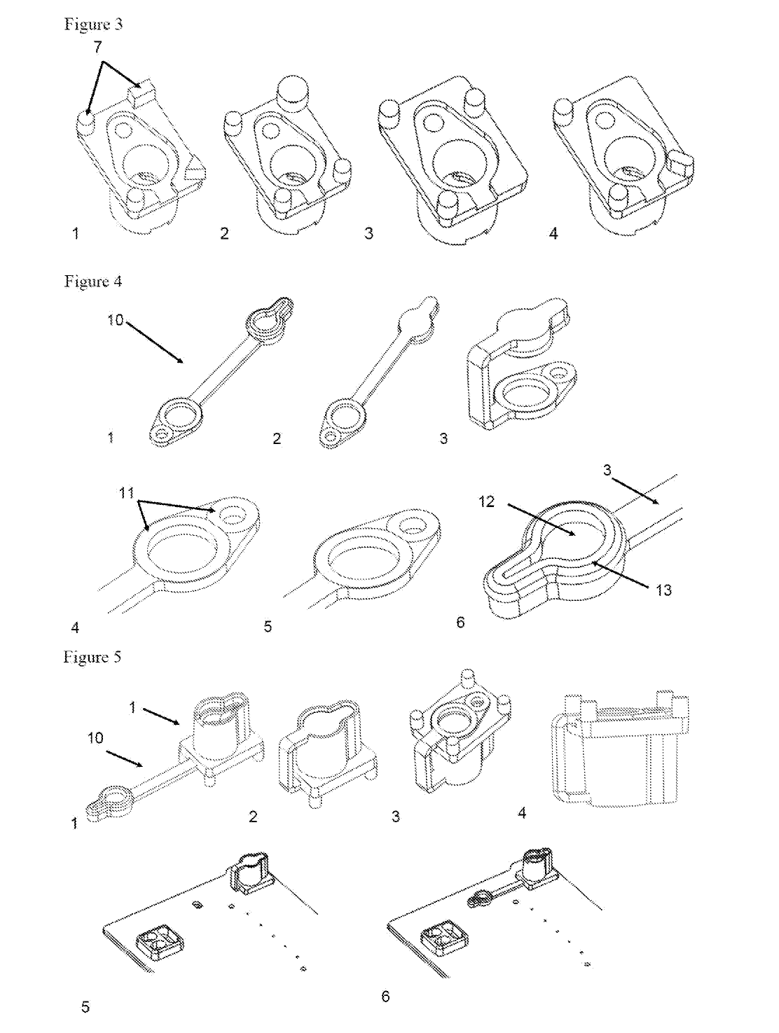

Example schematics of methods to key the alignment of the heat staked part are shown in FIG. 3. One or more or a combination of these methods can be used to heat stake the part into the microfluidic part. FIG. 3 shows a variety of different sizes and shapes of feet 7 that can be used to orient the component to the microfluidic card 5. FIG. 3 shows panel 1) Using multiple feet shapes to key the feature. The microfluidic card has corresponding holes that match the shapes such that the part aligns properly to the card in only one orientation; 2) Use of different sizes of the same feature type. 3) Use of non-symmetric feet; 4) Use of a kinematic mounting type structure.

In another embodiment, instead of using individualized feet, a major portion of the perimeter of the part is designed to extend through the microfluidic part (leaving a portion for the microchannels to pass).

C) Enclosure System that Starts as a Separate Component but then Becomes Integrated with the Port as Both a Gasket and a Lid

In some embodiments, a 1-piece gasket/enclosure system used in with swab port. The part is illustrated in FIG. 4. FIG. 4 shows a top and bottom view (1 and 2) of the 1 piece gasket/lid 10. In some embodiments, the gasket/lid 10 is made from a flexible material such as a silicone rubber and can fold/bend into place to fit around the main swab port body.

FIG. 4 also shows (lower panel) are raised edges 11 on the gasket section of the piece. The ridges are designed to focus the pressure and seal the swab port to the card. The lid/enclosure section 3 has a cavity 12 to allow for headspace on the broken swab and a ridge 13 that runs along the lateral perimeter to fit into small cavity inside the swab holder.

FIG. 5 illustrates how the gasket/enclosure piece 10 mates with the swab port 1. Until the two pieces are heat staked to the microfluidic card they are press fit/held together with friction. The lower picture highlights the ridges that protrude from the bottom of the sub-assembly. These ridges press against the microfluidic card during the heat staking to form a better seal. Once the swab port is heat staked to the microfluidic card the gasket it trapped and because the lid/enclosure section is also the same piece as the lid/enclosure the lid is also then permanently attached to the microfluidic card/swab port assembly. This provides the added benefit of not being able to lose the lid on the device.

In other embodiments, the swab port is attached to the microfluidics device using, for example, solvent bonding, adhesives, or double sided tape.

### D) Main Body—Swab Snapping/Reflux Port Concept for Out-of-Plane Mixing/Re-Circulation.

In some embodiments, the present disclosure provides features that allow a user to place a swab inside the swab port and snap off the non-useful stem portion of the swab while fully containing the active portion of the swab. In some embodiments, the present disclosure provides methodology allowing liquid to be refluxed over/through the swab in a controlled manner.

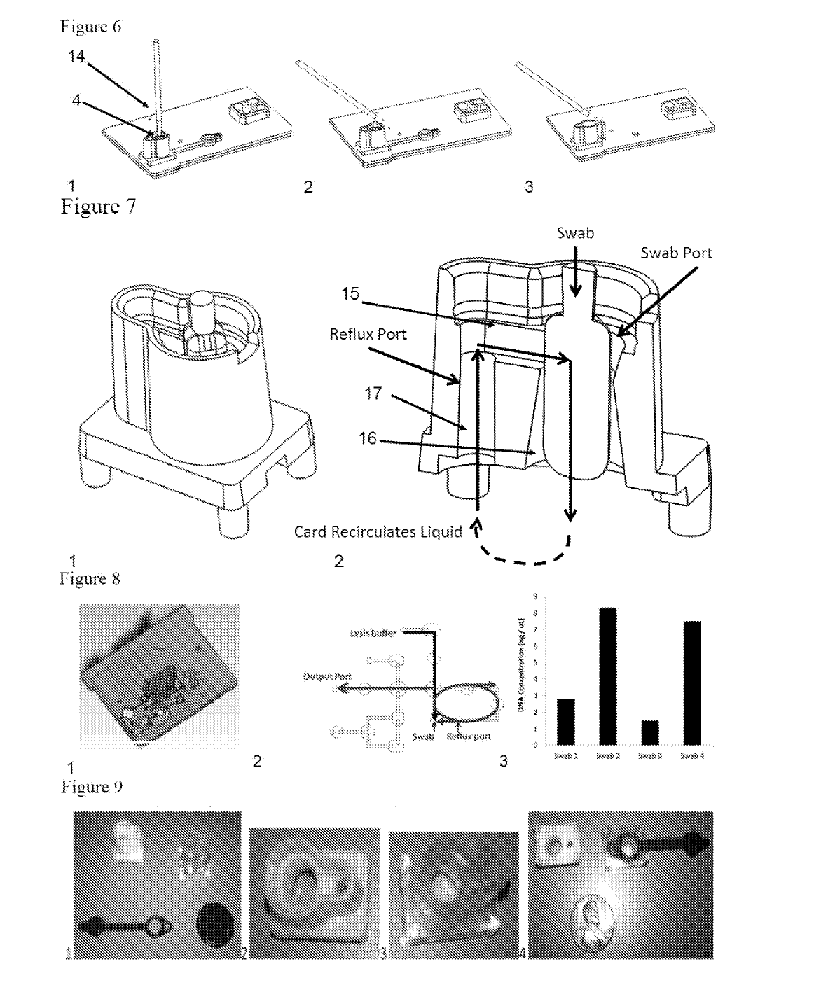

FIG. 6 illustrates placing a swab 14 into the swab insertion component 4 and then the main stem of the swab is snapped off, retaining the portion of the swab containing sample inside the swab port. A feature aiding the snapping process is the necking 15 pictured in the FIG. 7. A larger cavity 16 for the swab is provided in the bottom of the swab insertion component 4. This cavity tapers towards the top where it is slightly larger than the stem of the swab to form a bottle neck. This feature allows the swab to be pushed in with modest effort yet retain the swab unless a user specifically tries to pull it back out. This neck 15 also serves as a point to concentrate force on the stem. When the user applies lateral force to the entire swab the swab snaps at the neck thus leaving the useful part of the swab in the port and removing the long stem that would otherwise be difficult to seal and or load into instrumentation.

FIG. 7 illustrates a reflux port 17 next to the swab port. A sample fluid flow path is illustrated in FIG. 7 (the fluid path direction can also be reversed). The reflux port 17 is an empty vertical column that acts as a conduit or channel that connects the bottom of the swab to the top of the swab. Fluid can then be pumped up through the reflux port where it crosses over to the swab port through a horizontal channel to the top of the swab. That volume of liquid then gets pulled down over the swab surface and also through the swab (liquid permeates inside the swab) and then goes down into the microfluidic card where the liquid then gets pushed back up through the reflux port. The process is repeated several times (or several column volumes worth) and the interaction of the liquid with the swab and any biological materials on the swab is greatly enhanced. For instance, in some embodiments, this process aids in removal of cellular material from swabs and enhances cell lysis provided the liquid solution is a lysis buffer. It also aids in increasing recovery of biological materials (e.g., nucleic acids, amino acids, fats, oils, metabolites and the like) from forensic swabs and also for clinical swabs (nasal/throat/wound). Embodiments of the disclosure also find use in non-biological applications such as, for example, the enhanced recovery of explosive residues. The reflux port does not require the use of a lid/enclosure system, but they can be used together (e.g., to reduce risk of spillage and cross contamination). Additionally, the device does not require a swab for its reflux function. For example, in some embodiments, the device is loaded with a sample (e.g. whole blood) and the blood is refluxed with lysis buffer. The swab port is not limited to a particular sample or reflux port size. In some embodiments, the swab port holds ˜300 total uL of liquid without the swab present; however smaller and larger volumes are specifically contemplated

FIG. 8 shows an additional schematic of recirculation of liquid through a reflux port.

### II. The Device in Use

FIG. 8 shows a schematic of the device of embodiments of the present invention in use. The far right panel of FIG. 8 demonstrates DNA concentrations obtained were between 15-90 ng/uL which is greater than or equal to traditional bench-scale swab processing.

During experiments conducted during development of embodiments described herein liquids were circulated through a swab port. The swab was placed into the swab port and the majority of the swab stem was snapped (leaving the swabbing part of the swab in the column). Food coloring was microfluidically pumped to the swab and reflux ports to demonstrate that the columns are fluidically separated except for a cut notch at the top and through microfluidics at the bottom. The microfluidic device design is useful to pump to both the swab and reflux port at the same time from a liquid reservoir.

### A) Assays

In some embodiments, the devices, systems and kits describe herein find use in analysis and detection assays. The swab port and microfluidic devices describe herein find use in the detection and analysis of biological (e.g., nucleic acid, amino acid, fat, lipid, metabolite, small molecule) and chemical (e.g., environmental or warfare chemicals) analytes.

The microfluidic devices find use in a variety of assays including but not limited to, nucleic acid amplification, hybridization assays, immunoassays, chemical assays and the like.

In some embodiments, amplified analytes are further detected using a suitable technique. For example, in some embodiments, base compositions of amplification products are determined from detected molecular masses in order to identify nucleic acid analytes. In these embodiments, base compositions are typically correlated with the identity of an organismal source, genotype, or other attribute of the corresponding template nucleic acids in a given sample. Databases with base compositions and other information useful in these processes are also typically included in these systems. Suitable software and related aspects, e.g., for determining base compositions from detected molecular masses and for performing other aspects of base composition analysis are commercially available from Ibis Biosciences, Inc. (Carlsbad, Calif., U.S.A.).

Particular embodiments of molecular mass-based detection methods and other aspects that are optionally adapted for use with the systems described herein are described in various patents and patent applications, including, for example, U.S. Pat. Nos. 7,108,974; 7,217,510; 7,226,739; 7,255,992; 7,312,036; and 7,339,051; and US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; WO2002/070664; WO2003/001976; WO2003/100035; WO2004/009849; WO2004/052175; WO2004/053076; WO2004/053141; WO2004/053164; WO2004/060278; WO2004/093644; WO 2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; and WO2007/100397; WO2007/118222, which are each incorporated by reference as if fully set forth herein.

Exemplary molecular mass-based analytical methods and other aspects of use in the systems described herein are also described in, e.g., Ecker et al. (2005) “The Microbial Rosetta Stone Database: A compilation of global and emerging infectious microorganisms and bioterrorist threat agents” BMC Microbiology 5(1):19; Ecker et al. (2006) “The Ibis T5000 Universal Biosensor: An Automated Platform for Pathogen Identification and Strain Typing” JALA 6(11):341-351; Ecker et al. (2006) “Identification of Acinetobacter species and genotyping of Acinetobacter baumannii by multilocus PCR and mass spectrometry” J Clin Microbiol. 44(8):2921-32; Ecker et al. (2005) “Rapid identification and strain-typing of respiratory pathogens for epidemic surveillance” Proc Natl Acad Sci USA. 102(22):8012-7; Hannis et al. (2008) “High-resolution genotyping of Campylobacter species by use of PCR and high-throughput mass spectrometry” J Clin Microbiol. 46(4):1220-5; Blyn et al. (2008) “Rapid detection and molecular serotyping of adenovirus by use of PCR followed by electrospray ionization mass spectrometry” J Clin Microbiol. 46(2):644-51; Sampath et al. (2007) “Global surveillance of emerging Influenza virus genotypes by mass spectrometry” PLoS ONE 2(5):e489; Sampath et al. (2007) “Rapid identification of emerging infectious agents using PCR and electrospray ionization mass spectrometry” Ann N Y Acad Sci. 1102:109-20; Hall et al. (2005) “Base composition analysis of human mitochondrial DNA using electrospray ionization mass spectrometry: a novel tool for the identification and differentiation of humans” Anal Biochem. 344(1):53-69; Hofstadler et al. (2003) “A highly efficient and automated method of purifying and desalting PCR products for analysis by electrospray ionization mass spectrometry” Anal Biochem. 316:50-57; Hofstadler et al. (2006) “Selective ion filtering by digital thresholding: A method to unwind complex ESI-mass spectra and eliminate signals from low molecular weight chemical noise” Anal Chem. 78(2):372-378; and Hofstadler et al. (2005) “TIGER: The Universal Biosensor” Int J Mass Spectrom. 242(1):23-41, which are each incorporated by reference.

In addition to the molecular mass and base composition analyses referred to above, essentially any other nucleic acid amplification technological process is also optionally adapted for use in the systems of the invention. Other exemplary uses of the systems and other aspects of the invention include immunoassays, cell culturing, cell-based assays, compound library screening, and chemical synthesis, among many others. Many of these as well as other exemplary applications of use in the systems of the invention are also described in, e.g., Current Protocols in Molecular Biology, Volumes I, II, and III, 1997 (F. M. Ausubel ed.); Perbal, 1984, A Practical Guide to Molecular Cloning; the series, Methods in Enzymology (Academic Press, Inc.); Sambrook et al., 2001, Molecular Cloning: A Laboratory Manual, Third Edition, Cold Spring Harbor Laboratory Press, Cold Spring Harbor, N.Y.; Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Berger and Kimmel, Guide to Molecular Cloning Techniques, Methods in Enzymology volume 152 Academic Press, Inc., San Diego, Calif. (Berger), DNA Cloning: A Practical Approach, Volumes I and II, 1985 (D. N. Glover ed.); Immobilized Cells and Enzymes, 1986 (IRL Press); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively), which are each incorporated by reference.

### B) Kits and Systems

In some embodiments, the swab ports and related components are provided in kits. To illustrate, in some embodiments, kits include only swab ports, whereas in other exemplary embodiments kits also include lids, gaskets, microfluidic devices, etc. The material included in a given kit typically depends on the intended purpose of the devices (e.g., for use in a nucleic acid or protein purification process, for use in a cell culture process or screening application, for use in a painting or printing application, for use in chemical synthetic processes, etc.). Accordingly, non-limiting examples of materials optionally included in kits are magnetically responsive particles (e.g., magnetically responsive beads, etc.), water, solvents, buffers, reagents, cell culture media, cells, paint, ink, biopolymers (e.g., nucleic acids, polypeptides, etc.), solid supports (e.g., controlled pore glass (CPG), etc.), and the like. Kits typically also include instructions for using the devices and systems described herein. In addition, kits also generally include packaging for containing the devices and/or the instructions.

Kits are typically provided in response to receiving an order from a customer. Orders are received through a variety of mechanisms including, e.g., via a personal appearance by the customer or an agent thereof, via a postal or other delivery service (e.g., a common carrier), via a telephonic communication, via an email communication or another electronic medium, or any other suitable method. Further, kits are generally supplied or provided to customers (e.g., in exchange for a form of payment) by any suitable method, including via a personal appearance by the customer or an agent thereof, via a postal or other delivery service, such as a common carrier, or the like.

In some embodiments, the swab ports and microfluidic devices are provided as part of a system. In some embodiments, systems comprise swab ports (e.g., provided in the form of a kit) and microfluidic devices or other assay devices. In some embodiments, systems further comprise sample handling components and automated assay components.

Sample handling components and/or other system components is/are generally coupled to an appropriately programmed processor, computer, digital device, or other logic device or information appliance (e.g., including an analog to digital or digital to analog converter as needed), which functions to instruct the operation of these instruments in accordance with preprogrammed or user input instructions (e.g., addition of reagents, transfer of reagents to additional components, fluid volumes to be conveyed, etc.), receive data and information from these instruments, and interpret, manipulate and report this information to the user.

A controller or computer optionally includes a monitor which is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming.

The computer can be, e.g., a PC (Intel x86 or Pentium chip-compatible DOS™, OS2™, WINDOWS™, WINDOWS NT™, WINDOWS98™, WINDOWS2000™, WINDOWS XP™, WINDOWS Vista™, LINUX-based machine, a MACINTOSH™, Power PC, or a UNIX-based (e.g., SUN™ work station) machine) or other common commercially available computer which is known to one of skill. Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention. Software for performing, e.g., sample handling, assay detection, and data deconvolution is optionally constructed by one of skill using a standard programming language such as Visual basic, C, C++, Fortran, Basic, Java, or the like.

In some embodiments, systems include detection components configured to detect one or more detectable signals or parameters from a given process, e.g., from assays carried out in microfluidic devices. In some embodiments, systems are configured to detect detectable signals or parameters that are upstream and/or downstream of a given assay. Suitable signal detectors that are optionally utilized in these systems detect, e.g., pH, temperature, pressure, density, salinity, conductivity, fluid level, radioactivity, luminescence, fluorescence, phosphorescence, molecular mass, emission, transmission, absorbance, and/or the like. In some embodiments, the detector monitors a plurality of signals, which correspond in position to “real time” results. Example detectors or sensors include PMTs, CCDs, intensified CCDs, photodiodes, avalanche photodiodes, optical sensors, scanning detectors, or the like. Each of these as well as other types of sensors is optionally readily incorporated into the systems described herein. The detector optionally moves relative to assay devices or stations, sample containers or other assay components, or alternatively, assay devices or stations, sample containers or other assay components move relative to the detector. Optionally, the systems include multiple detectors.

The detector optionally includes or is operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of a few or even a single communication port for transmitting information between system components. Detection components that are optionally included in the systems of the invention are described further in, e.g., Skoog et al., Principles of Instrumental Analysis, 6^{th} Ed., Brooks Cole (2006) and Currell, Analytical Instrumentation: Performance Characteristics and Quality, John Wiley & Sons, Inc. (2000), which are both incorporated by reference.

The systems optionally also include at least one robotic translocation or gripping component that is structured to grip and translocate swab ports or microfluidic devices or other components between components of the stations or systems and/or between the stations or systems and other locations (e.g., other work stations, etc.). A variety of available robotic elements (robotic rms, movable platforms, etc.) can be used or modified for use with these systems, which robotic elements are typically operably connected to controllers that control their movement and other functions.

Suitable linear motion components, motors, and motor drives are generally available from many different commercial suppliers including, e.g., Techno-Isel Linear Motion Systems (New Hyde Park, N.Y., U.S.A.), NC Servo Technology Corp. (Westland, Mich., USA), Enprotech Automation Services (Ann Arbor, Mich., U.S.A.), Yaskawa Electric America, Inc. (Waukegan, Ill., U.S.A.), ISL Products International, Ltd. (Syosset, N.Y., U.S.A.), AMK Drives & Controls, Inc. (Richmond, Va., U.S.A.), Aerotech, Inc. (Pittsburgh, Pa., U.S.A.), HD Systems Inc. (Hauppauge, N.Y., U.S.A.), and the like. Additional detail relating to motors and motor drives are described in, e.g., Polka, Motors and Drives, ISA (2002) and Hendershot et al., Design of Brushless Permanent-Magnet Motors, Magna Physics Publishing (1994), which are both incorporated by reference. Microplate handling components are also described in, e.g., Attorney Docket No. DIBIS-0116US.L, entitled “MICROPLATE HANDLING SYSTEMS AND RELATED COMPUTER PROGRAM PRODUCTS AND METHODS” filed Sep. 16, 2008 by Hofstadler et al., which is incorporated by reference in its entirety.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A swab port device, comprising:
at least one body structure comprising one or more surfaces that define a first cavity having upper and lower portions sized to accept a sample collection swab; and a second cavity having upper and lower portion; and
a lid configured to seal said first and second cavities.

2. The device of claim 1, wherein said device further comprises at least a portion of a sample collection swab.

3. The device of claim 1, wherein said first and second cavities are in fluid communication.

4. The device of claim 1, wherein said first cavity comprises a volume capacity of about 300 μL.

5. The device of claim 1, wherein said body structure further comprises a plurality of protrusions.

6. The device of claim 5, wherein said protrusions comprise a plurality of protrusions of different sizes or shapes.

7. The device of claim 5, wherein said protrusions are configured to align said device to holes in an analysis device.

8. The device of claim 1, wherein said lid comprises a lid sealing component and a gasket component.

9. The device of claim 1, wherein said first cavity comprises a neck.

10. The device of claim 1, wherein said lid is integrated into said swab port device.

11. The device of claim 1, wherein said first cavity has an interior surface comprising one or more protrusions configured to assist in the removal of material from an inserted swab.

12. A system, comprising:
a) the device of claim 1; and
b) an assay component in communication with said device.

13. The system of claim 12, wherein said protrusions of said device are inserted in holes in said assay component.

14. The system of claim 13, wherein said protrusions are heat sealed to said assay component.

15. The system of claim 12, wherein said assay component is a microfluidic device.

16. The system of claim 12, wherein said system further comprises a sample analysis component operably linked to said assay component.

17. (canceled)

18. A method, comprising:
a) contacting the system of claim 12 with a swab comprising a sample; and
b) circulating liquid contained in said second cavity through said first cavity.

19. The method of claim 18, further comprising the step of breaking off the end of said swab such that the portion of said swab containing said sample remains in the first cavity of said device

20. The method of claim 18, wherein said sample is transferred to said liquid.

21. The method of claim 20, further comprising the step of contacting said liquid with said assay component.

22. The method of claim 21, further comprising the step of identifying an analyte in said sample.

23. The method of claim 21, wherein said analyte is selected form the group consisting of a nucleic acid, an amino acid, a lipid, a metabolite, and a chemical.

24-26. (canceled)
